(19) United States
(12) Patent Application Publication
(10) Pub. No.: US 2021/0331129 A1
(43) Pub. Date: Oct. 28, 2021

(54) MOTORIZED MIXING DEVICE FOR USE WITH MEDICAL AGENTS

(71) Applicant: Covidien LP, Mansfield, MA (US)

(72) Inventors: Junmin GUO, Shanghai (CN); Shichang WEN, Shanghai (CN); Quan ZHANG, Shanghai (CN); Dongliang SONG, Shanghai (CN)

(21) Appl. No.: 17/309,063

(22) PCT Filed: Apr. 20, 2020
(86) PCT No.: PCT/CN2020/085631

§ 371 Date: Apr. 19, 2021

(51) Int. Cl. B01F 15/02; B01F 11/00; B01F 13/00; B01F 15/00; A61M 5/315

(52) U.S. Cl. CPC B01F 15/0278; B01F 11/0054; B01F 2215/0034; B01F 15/00538; A61M 5/31596; B01F 13/0023

(57) ABSTRACT

A device for mixing a medical agent and associated systems and methods are disclosed herein. In some embodiments, the device comprises (i) a housing defining a chamber configured to contain a fluid and having a longitudinal axis, (ii) a plunger assembly at least partially disposed within the chamber, the plunger assembly comprising a plunger and a stirring member extending along the longitudinal axis, and (iii) an actuating assembly operably coupled to the stirring member. The actuating assembly can have an active state in which the actuating assembly causes a distal end portion of the stirring member to rotate at a predetermined speed.

### TECHNICAL FIELD

The present technology relates to systems, devices, and methods for providing and/or mixing a medical agent.

### BACKGROUND

Fluid medical agents are commonly used in the medical field for multiple purposes, including for temporary or permanent embolization or occlusion of blood vessels. Medical agents designed for these and other purposes are often prepared just prior to usage (e.g., delivery to a patient) because the functionality of the medical agent can alter over time to become less effective. For example, solutes of the medical agent can undesirably precipitate over time after mixing, and/or the visualization quality of the agent can undesirably degrade over time after mixing. As a result, healthcare professional often need to spend extra time mixing or remixing the medical agents immediately prior to usage. However, such extra time can be detrimental to the patient being treated. For example, the mixing or remixing of the medical agent often occurs during surgery and thus prolongs the length of the surgery, thereby increasing risk for the patient. For these and other reasons, there is a need for innovation in the mixing of medical agents.

### SUMMARY

The subject technology is illustrated, for example, according to various aspects described below, including with reference to FIGS. 1A-7. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., Clause 1, Clause 21, etc. The other clauses can be presented in a similar manner

Clause 1. A device for mixing a medical agent, the device comprising:

a housing defining a chamber configured to contain a fluid and having a longitudinal axis;

a plunger assembly configured to be received by the chamber, the plunger assembly comprising a plunger and a stirring member extending along the longitudinal axis, the plunger being rotatably coupled to the stirring member, the stirring member having a distal portion disposed within the chamber of the housing; and

an actuating assembly operably coupled to the stirring member, the actuating assembly having an active state in which the actuating assembly causes the distal end portion of the stirring member to rotate at a predetermined speed.

Clause 2. The device of any one of the clauses herein, wherein the actuating assembly comprises a motor disposed along the longitudinal axis, and wherein rotation of the motor causes the distal end portion of the stirring member to rotate.

Clause 3. The device of any one of the clauses herein, wherein the motor is a step motor configured to rotate at a plurality of speeds including a first speed, and a second speed different than the first speed.

Clause 4. The device of any one of the clauses herein, further comprising a coupling member coupled directly to the actuating assembly and the stirring member such that rotation of the actuating assembly causes corresponding rotation of at least one of the coupling member or the stirring member.

Clause 5. The device of any one of the clauses herein, further comprising a biasing member disposed between the plunger and the actuating assembly, the biasing member urging the plunger distally toward the housing.

Clause 6. The device of any one of the clauses herein, wherein the plunger is movable relative to the housing from a first position adjacent a distal portion of the chamber to a second position spaced apart from the distal portion of the chamber, wherein, when the biasing member is in a first state when the plunger is in the first position and a second state when the plunger is in the second position, and wherein the biasing member in the second state is more compressed than the biasing member in the first state.

Clause 7. The device of any one of the clauses herein, further comprising a coupling member having a proximal end and a distal end opposite the proximal end, the coupling member receiving a portion of the actuating assembly at the proximal end and a portion of the stirring member at the distal end, wherein the biasing member is disposed between the coupling member and the plunger such that the biasing members acts against the coupling member and the plunger to urge the plunger distally.

Clause 8. The device of any one of the clauses herein, wherein the stirring member is longitudinally moveable relative to the plunger.

Clause 9. The device of any one of the clauses herein, wherein the plunger includes a slot extending along the longitudinal axis, and wherein the stirring member extends through the slot and is longitudinally moveable relative to the plunger.

Clause 10. The device of any one of the clauses herein, further comprising an exit port at a distal end of the housing, the exit port being configured to be coupled to a connection assembly via luer lock connection fittings.

Clause 11. The device of any one of the clauses herein, wherein the stirring member comprises a flared portion at the distal portion of the stirring member, the flare portion extending at an angle relative to the longitudinal axis.

Clause 12. A medical device system, comprising:

a mixing device including—
a housing defining a chamber configured to contain a fluid and having a longitudinal axis;
a plunger assembly configured to be received by the chamber, the plunger assembly comprising a plunger and a stirring member extending along the longitudinal axis, the plunger being rotatably coupled to the plunger, the stirring member having a distal portion disposed within the chamber of the housing; and
an actuating assembly operably coupled to the stirring member, the actuating assembly having an active state in which the actuating assembly causes the distal end portion of the stirring member to rotate at a predetermined speed.

Clause 13. The system of any one of the clauses herein, further comprising a connection assembly including a first port coupled to an exit port of the housing, and a second port exposed to the atmosphere, wherein a flow path between the first and second ports is configured to release trapped air within the chamber.

Clause 14. The system of any one of the clauses herein, further comprising a syringe and a connection assembly, the connection assembly including a first port coupled to an exit port of the housing, and a second port coupled to the syringe, wherein the chamber of the device and the syringe are in fluid communication with one another via a flow path extending between the first and second ports.

Clause 15. The system of any one of the clauses herein, further comprising a connection assembly including:

a flow control element,

a first port coupled to an exit port of the housing,

a second port configured to be exposed to the atmosphere, and

a third port configured to be coupled to a syringe,

wherein movement of the flow control element creates a flow path between the first port and one of the second port or the third port.

Clause 16. A method for mixing a medical agent, the method comprising:

providing a mixing device including—
a housing defining a chamber containing a fluid and having a longitudinal axis;
a plunger assembly at least partially disposed within the chamber, the plunger assembly including a plunger and a stirring member rotatably coupled to the plunger, the stirring member disposed within the chamber of the housing such that a distal portion of the stirring member is proximate the fluid; and
an actuating assembly operably coupled to the stirring member, the actuating assembly having an active state in which the actuating assembly causes the distal end portion of the stirring member to rotate at a predetermined speed

operating the actuating assembly such that the stirring member mixes the fluid.

Clause 17. The method of any one of the clauses herein, wherein the mixing device is in a first configuration in which the plunger is positioned at a distal portion of the chamber, the method further comprising urging the plunger proximally along the longitudinal axis toward the actuating assembly, thereby compressing a biasing member of the device.

Clause 18. The method of any one of the clauses herein, wherein the actuating assembly comprises a motor, and wherein operating the actuating assembly comprises activating the motor to rotate at a predetermined speed for a predetermined period of time.

Clause 19. The method of any one of the clauses herein, further comprising:

coupling a first port of a connection assembly to an exit port of the housing; and

enabling trapped air in the chamber to be released via a second port of the connection assembly.

Clause 20. The method of any one of the clauses herein, further comprising:

coupling a first port of a connection assembly to an exit port of the housing;

coupling a syringe to a second port of the connection assembly; and

transferring the fluid from the chamber to the syringe via a path include the first and second ports of the connection assembly.

Clause 21. A mixing device for use with medical agents, the device comprising:

a housing having a chamber configured to contain a fluid, and a longitudinal axis;

a plunger at least partially disposed within the chamber of the housing such that the plunger is movable along the longitudinal axis relative to the housing;

a rotatable stirring member having a distal end portion disposed within the chamber of the housing, the stirring member extending along the longitudinal axis; and

an actuating assembly operably coupled to the stirring member, the actuating assembly having an active state in which the actuating assembly causes the distal end portion of the stirring member to rotate.

Clause 22. The device of any one of the clauses herein, wherein the actuating assembly comprises a motor operably coupled to the stirring member, the motor being configured to rotate the stirring member at a plurality of different speeds.

Clause 23. The device of any one of the clauses herein, wherein the actuating assembly comprises a motor operably coupled to the stirring member, the motor being configured to rotate the stirring member in a first direction and a second direction opposite the first direction.

Clause 24. The device of any one of the clauses herein, wherein the actuating assembly is proximal to at least one of the housing, the plunger, or the stirring member.

Clause 25. The device of any one of the clauses herein, wherein the motor is generally disposed along the longitudinal axis.

Clause 26. The device of any one of the clauses herein, wherein the actuating assembly is configured to rotate at a speed based on the fluid.

Clause 27. The device of any one of the clauses herein, wherein the actuating assembly is configured to rotate at a speed based on an upper temperature limit of the fluid.

Clause 28. The device of any one of the clauses herein, further comprising a biasing member disposed along the longitudinal axis between the actuating assembly and the plunger.

Clause 29. The device of any one of the clauses herein, wherein the biasing member urges the plunger in a distal direction.

Clause 30. The device of any one of the clauses herein, wherein the biasing member is configured to transition from a first state to a second state more compressed than the first state, and wherein transitioning from the first state to the second state causes the plunger to move proximally toward the actuating assembly.

Clause 31. The device of any one of the clauses herein, wherein the biasing member is configured to transition from a first state to a second state more compressed than the first state, wherein transitioning from the first state to the second state causes the plunger to move proximally relative to the stirring member.

Clause 32. The device of any one of the clauses herein, wherein the biasing member is configured to transition from a first state to a second state less compressed than the first state, wherein transitioning from the first state to the second state causes the plunger to move distally.

Clause 33. The device of any one of the clauses herein, wherein the biasing member is a spring.

Clause 34. The device of any one of the clauses herein, wherein the stirring member is directly coupled to a motor of the actuating assembly.

Clause 35. The device of any one of the clauses herein, wherein the stirring member is movable along the longitudinal axis relative to at least one of the housing, the chamber, or the plunger.

Clause 36. The device of any one of the clauses herein, wherein the stirring member is rotatably coupled to the plunger.

Clause 37. The device of any one of the clauses herein, wherein the distal end portion of the stirring member comprises a flared portion or wings extending in an axial direction angled relative to the longitudinal axis.

Clause 38. The device of any one of the clauses herein, wherein the chamber includes a distal end portion having a first angled surface, and wherein the distal end portion of the stirring member has a second angled surface corresponding to the first angled surface.

Clause 39. The device of any one of the clauses herein, wherein the plunger has a slot through which the stirring member is disposed.

Clause 40. The device of any one of the clauses herein, wherein a distal end portion of the slot has a shape corresponding to that of the flared portion or wings of the distal end portion of the stirring member.

Clause 41. The device of any one of the clauses herein, wherein the plunger is disposed within the chamber such that the plunger provides an air-tight seal for the chamber.

Clause 42. The device of any one of the clauses herein, wherein the plunger comprises one or more rings configured to provide an air-tight seal for the chamber.

Clause 43. The device of any one of the clauses herein, wherein a distal end portion of the plunger includes a conical surface corresponding to a surface at a distal end portion of the chamber of the housing.

Clause 44. The device of any one of the clauses herein, wherein at least one of the housing or the plunger comprises silicon.

Clause 45. The device of any one of the clauses herein, further comprising a coupling member disposed between the actuating assembly and the plunger, the coupling member including a recess configured to receive a proximal end portion of the stirring member.

Clause 46. The device of any one of the clauses herein, further comprising a coupling member directly coupling the actuator assembly to the stirring member such that movement of the actuator assembly causes corresponding movement of the coupling member and the stirring member.

Clause 47. The device of any one of the clauses herein, further comprising (i) a coupling member disposed between the actuating assembly and the plunger, and (ii) a biasing member disposed around a portion of the coupling member and urging the coupling member proximally.

Clause 48. The device of any one of the clauses herein, further comprising a luer lock connection fitting at a distal end portion of the housing, the connection fitting disposed adjacent or around an exit port of the housing.

Clause 49. The device of any one of the clauses herein, wherein the fluid is a medical agent.

Clause 50. The device of ally one of the clauses herein, wherein the medical agent comprises an agent configured to cause temporary or permanent embolization or occlusion within a blood vessel of a patient.

Clause 51. The device of any one of the clauses herein, wherein the medical agent comprises at least one of a liquid agent, sclerosant, polymer, ethanol, sodium tetradecyl sulfate, N-butyl cyanoacrylate, ethylene vinyl alcohol, polyvinyl alcohol, dimethyl sulfoxide, tantalum powder, cellulose, collagen and/or gelatin.

Clause 52. The device of any one of the clauses herein, wherein the fluid comprises ethylene vinyl alcohol copolymer, dimethyl sulfoxide and/or tantalum powder.

Clause 53. A medical device system, comprising:

the mixing device of any one of the clauses herein; and

a connection assembly comprising a plurality of ports including (i) a first port operably coupled to an exit port of the mixing device, and (ii) a second port able to fluidly communicate with the first port.

Clause 54. The system of any one of the clauses herein, further comprising a syringe operably coupled to the second port of the connection assembly.

Clause 55. The system of any one of the clauses herein, wherein the syringe includes a barrel defining a chamber, and a plunger slidably coupled to the barrel and moveable within the chamber.

Clause 56. The system of any one of the clauses herein, wherein the connection assembly comprises a third port able to fluidly communicate with the first port, wherein the third port is configured to release trapped air in the fluid contained in the chamber of the housing.

Clause 57. The system of any one of the clauses herein, wherein the connection assembly includes male or female connection fittings at the first port and/or the second port.

Clause 58. The system of any one of the clauses herein, further comprising a flow control element disposed between the first and second ports, the flow control element being movable to control fluid flow from the first port to (i) the second port and/or (ii) the third port.

Clause 59. A method of operating a mixing device, the method comprising:

providing the mixing device of any one of the clauses herein; and

operating the actuating assembly, thereby causing the distal end portion of the stirring member of the mixing device to rotate and mix the fluid contained in the chamber of the housing of the mixing device.

Clause 60. The method of any one of the Clauses herein, further comprising coupling a connection assembly to an exit port of the mixing device such that the connection assembly and mixing device are in fluid communication.

Clause 61. The method of any one of the Clauses herein, further comprising adjusting a flow control element of the connection assembly to create a flow path from the exit port to a port of the connection assembly, thereby enabling air to escape from the fluid via a path including the exit port of the mixing device and the port of connection assembly.

Clause 62. The method of any one of the Clauses herein, wherein the port of the connection assembly is a first port, the method further comprising coupling a syringe to a second port of the connection assembly.

Clause 63. The method of any one of the Clauses herein, further comprising adjusting the flow control element of the connection assembly to create a flow path from the exit port of the mixing device to the second port of the connection assembly.

Clause 64. The method of any one of the Clauses herein, wherein adjusting the flow control element to create the flow path from the exit port to the second port comprises adjusting the flow control element to create the flow path from the exit port to the second port after releasing trapped air from the fluid via the first port of the connection assembly.

Clause 65. The method of any one of the Clauses herein, further comprising transferring the fluid from the mixing device to the syringe via the connection assembly.

Clause 66. The method of any one of the Clauses herein, further comprising drawing the fluid into the chamber of the housing by moving the plunger proximally relative the housing.

Clause 67. The method of any one of the Clauses herein, wherein urging the plunger proximally causes the biasing member of the mixing device to compress.

Clause 68. The method of any one of the Clauses herein, wherein operating the actuating assembly comprises operating the actuating assembly at one of a plurality of speeds the actuating assembly is capable of operating at.

Clause 69. The method of any one of the Clauses herein, wherein operating the actuating assembly comprises operating the actuating assembly at a first speed for a predetermined first period of time and then operating the actuating assembly at a second, different speed for a predetermined period of time.

Clause 70. The method of any one of the Clauses herein, wherein operating the actuating assembly occurs for a period of time no more than 20 minutes, 15 minutes, 10 minutes, 5 minutes, or 1 minute.

Clause 71. The method of any one of the Clauses herein, further comprising, disposing the mixing device in a cooling bath.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIGS. 1A, 1B, and 1C show perspective, side, and cross-sectional views respectively of a system for mixing medical agents, in accordance with embodiments of the present technology.

FIGS. 2A, 2B, and 2C show perspective, top, and side views respectively of a mixing device, in accordance with embodiments of the present technology.

FIGS. 3A, 3B, and 3C show exploded perspective, side, and cross-sectional views respectively of the mixing device shown in FIGS. 2A-2C.

FIGS. 4A and 4B show cross-sectional views of a mixing device in different configurations, in accordance with embodiments of the present technology.

FIGS. 5A and 5B show cross-sectional views of a mixing device, in accordance with embodiments of the present technology.

FIGS. 6 and 7 are flow diagrams of methods for mixing a medical agent, in accordance with embodiments of the present technology.

### DETAILED DESCRIPTION

### I. Overview

Fluid medical agents used in the medical field, e.g., for temporary or permanent embolization or occlusion of blood vessels, are often prepared just prior to usage (e.g., delivery to a patient via catheter) due to their decreased functionality and/or effectiveness over time. For example, solutes of the medical agents can begin to undesirably precipitate over time after mixing, and/or the visualization quality of the agents cam undesirably degrade over time after mixing. As a result, healthcare professionals often need to spend extra time mixing or remixing the medical agents prior to use, which can be detrimental to the patient being treated, e.g., by increasing the length of a surgery.

Embodiments of the present technology provide an improved ability to mix medical agents and can mitigate the risks previously described. For example, as explained in additional detail elsewhere herein, embodiments of the present technology can comprise a device for mixing a medical agent comprising (i) a housing defining a chamber configured to contain a fluid and having a longitudinal axis, (ii) a plunger assembly comprising a plunger and a stirring member extending along the longitudinal axis, and (iii) an actuating assembly operably coupled to the stirring member. The actuating assembly can have an active state in which the actuating assembly causes the distal end portion of the stirring member to rotate at a predetermined speed. By utilizing the actuating assembly, or a motor of the actuating assembly, the fluid contained in the chamber can be efficiently mixed, e.g., at a predetermined speed and/or a predetermined period of time. In doing so, healthcare professionals no longer need to spend extra time mixing or remixing the medical agents prior to usage and/or during surgery. Accordingly, in addition to ensuring a medical agent to be delivered to a patient is properly and/or efficiently mixed prior to usage, embodiments of the present technology can also help decrease the overall time of a surgery, and thereby generally decrease risk for the patient.

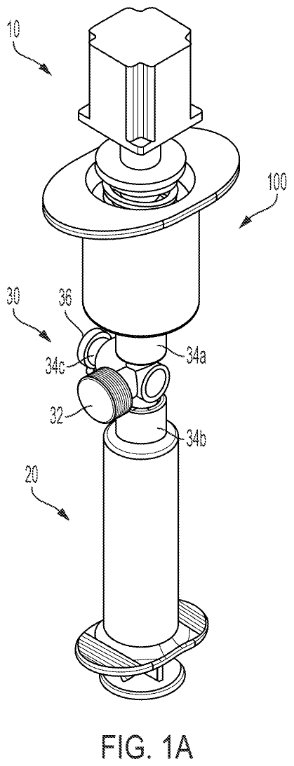

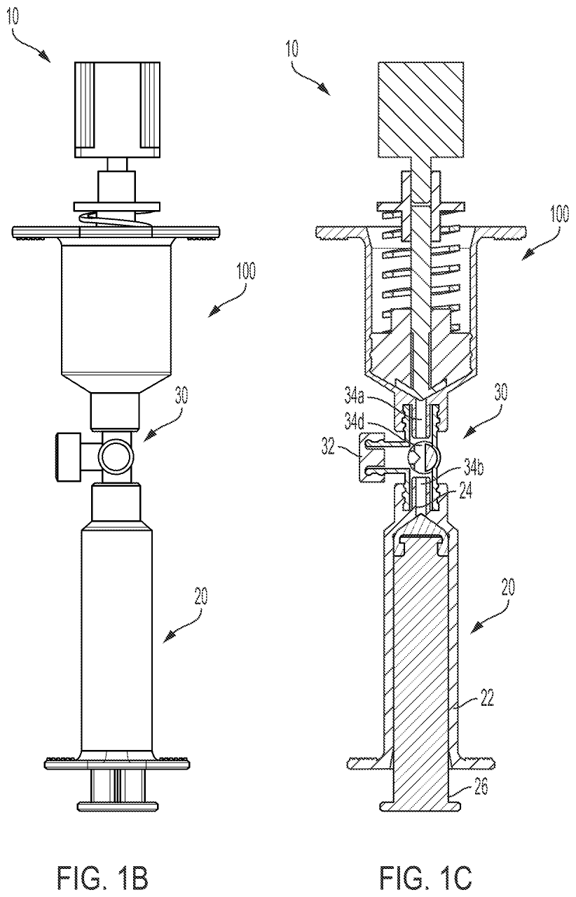

FIGS. 1A-7 depict embodiments of devices, systems and/or methods for mixing a fluid. Although many of the embodiments are described with respect to devices, systems, and methods for mixing medical agents, other applications and other embodiments in addition to those described herein are within the scope of the present technology. Further, embodiments of the present technology can have different configurations, components, and/or procedures than those shown or described herein. Moreover, embodiments of the present technology can have configurations, components, and/or procedures in addition to those shown or described herein and these and other embodiments may not have several of the configurations, components, and/or procedures shown or described herein without deviating from the present technology.

As used herein, the terms “distal” and “proximal” define a position or direction with respect to an operator or an operator's control device (e.g., a handle of a mixing device or syringe). For example, the terms, “distal” and “distally” refer to a position distant from or in a direction away from an operator or an operator's control device along the length of the device. In a related example, the terms “proximal” and “proximally” refer to a position near or in a direction toward an operator or an operator's control device along the length of the device. The headings provided herein are for convenience only and should not be construed as limiting the subject matter disclosed.

### II. Selected Devices, Systems, and Methods for Mixing Medical Agents

FIGS. 1A and 1B show perspective and side views respectively of a system 10 for mixing a medical agent, and FIG. 1C shows a cross-sectional view of the system 10 shown in FIG. 1B. Referring to FIGS. 1A-1C together, the system 10 can include a mixing device 100, a connection assembly 30 operably coupled to the mixing device 100 (e.g., at an exit port of the mixing device 100), and a syringe 20 operably coupled to the connection assembly 30. The connection assembly 30 can be disposed between the mixing device 100 and syringe 20, e.g., to enable fluid (e.g., a mixed medical agent) to be transferred from/to the mixing device 100 to/from the syringe 20 via the connection assembly 30. The syringe 20 can include a barrel or housing 22 (FIG. 1C), and a plunger 26 (FIG. 1C) configured to be received by the housing 22 such that the plunger 26 is slidably moveable along a longitudinal axis of the syringe 20. In some embodiments, the mixed medical agent may be provided from the mixing device 100 and/or syringe 20 to a catheter or related device or system for delivery to a target site of a patient. In some embodiments, the medical agent can comprise a liquid agent, sclerosant, polymer, ethanol, sodium tetradecyl sulfate, N-butyl cyanoacrylate, ethylene vinyl alcohol, polyvinyl alcohol, dimethyl sulfoxide, tantalum powder, cellulose, collagen and/or gelatin. In some embodiments, the medical agent can comprise the ONYX™ liquid embolic manufactured by Medtronic of Irvine, Calif., USA.

The connection assembly 30 can comprise a stopcock, and can include a plurality of ports configured to be in fluid communication with one another. For example, as shown in FIG. 1A, the connection assembly 30 can include a first port 34a coupled to the mixing device 100, a second port 34b coupled to the syringe 20, a third port 34c configured to be exposed to atmosphere, and a fourth (blank) port 34d. The connection assembly 30 can also include a flow control element 32 (e.g., a knob) that is movable to control fluid flow to, from, and/or between the different ports. For example, the flow control element 32 can have a first position creating a first flow path between the first and third ports 34a, 34c, a second position creating a second flow path between the second and third ports 34b, 34c, a third position creating a third flow path between the first and second ports 34a, 34b, and a fourth position such that no flow path exists between any of the first, second, and third ports 34a, 34b, 34c.

As an operational example, after a medical agent is mixed in the mixing device 100, the flow control element 32 may be operated to create the first flow path between the first and third ports 34a, 34c and thereby allow any trapped air in the mixed medical agent and/or chamber of the mixing device to be released to the atmosphere. Here, the mixing device 100 may be operated to urge the mixed medical agent in the mixing device 100 toward the connection assembly 30. Subsequently, the flow control element 32 may be operated to create the second flow path between the first and second ports 34a, 34b and thereby allow the mixed medical agent (with substantially no trapped air) to be transferred from the mixing device 100 to the syringe 20. Here, the mixing device 100 may be operated to urge the medical agent toward the connection assembly 30 and/or the syringe 20 may be operated (e.g., simultaneously operated with the mixing device 100) to urge the mixed medical agent toward the syringe 20 (e.g., by pulling the plunger 26 proximally). Subsequently, the flow control element 32 may be operated to the fourth position to close off all flow paths between the ports.

As shown in FIG. 1C, the connection assembly 30 may be coupled to each of the mixing device 100 and syringe 20 via connection fittings (e.g., male or female connection fittings). The connection fittings can be luer-lock type connection fittings, as well as other commonly used fittings provided in the art.

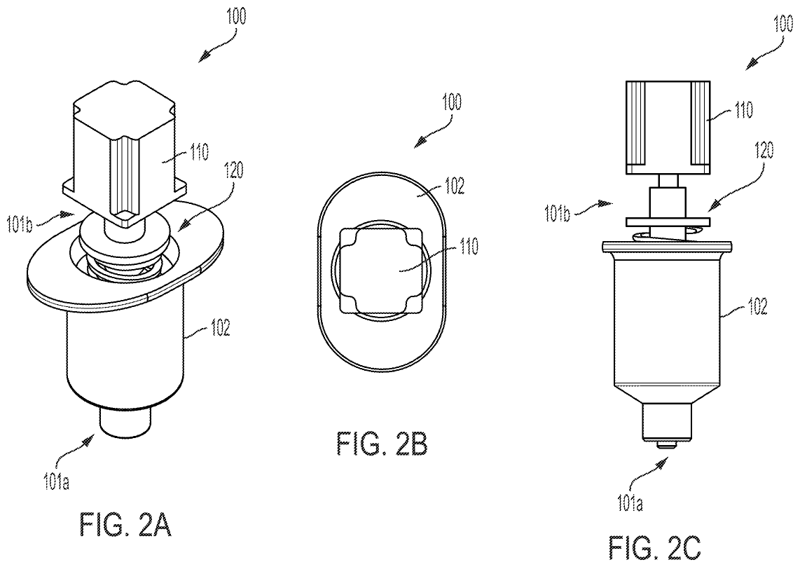

FIGS. 2A, 2B, and 2C show perspective, top, and side views respectively of the mixing device 100 shown in FIGS. 1A-1C. Referring to FIGS. 2A-2C together, the device 100 can include a distal end portion 101a, a proximal end portion 101b, a housing or barrel 102, a plunger assembly 120 coupled to and/or disposed at least partially within the housing 102, and an actuating assembly 110 (e.g., a motorized assembly) coupled to the plunger assembly 120. The actuating assembly 110 can be disposed at the proximal end portion 101b (e.g., the proximalmost end) of the device 100. In some embodiments, the device 100 shown in FIGS. 2A-2C and as described elsewhere herein may be provided as a kit in which individual components of the device 100 are uncoupled.

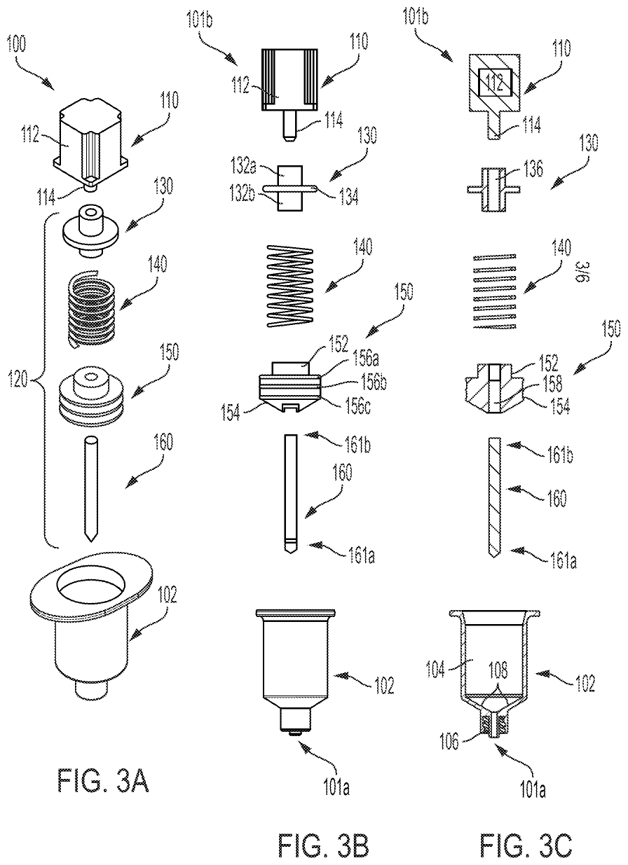

FIGS. 3A and 3B show exploded perspective and side views respectively of the device 100 shown in FIGS. 2A-2C, and FIG. 3C shows a cross-sectional exploded view of the device 100 shown in FIG. 3B. Referring to FIGS. 3A-3C together, the device 100 can include the actuating assembly 110, the plunger assembly 120 coupled to the actuating assembly 110, and the housing 102 surrounding at least part of the plunger assembly 120.

The actuating assembly 110 can include a motor and associated components 112 (collectively referred to as “motor 112”; shown schematically in FIG. 3C), including a gearbox, encoder (e.g., magnetic or optical encoder), bearings (e.g., sleeve or ball bearings), rotor coil, and/or associated sensors and electronics (e.g., for feedback purposes). For example, in some embodiments the sensors can include a temperature sensor (e.g., a thermocouple or resistance temperature detector (RTD)) configured to sense a temperature of a fluid in a chamber of the housing 102, e.g., to ensure the fluid does not rise above a predetermined temperature. Such a sensor may help ensure medical agents are not degraded (e.g., ensure proteins are not denatured). The motor 112 can include a stepper motor, a brushless direct current (DC) motor, or a brushed DC motor, and can be configured to rotate at a plurality of speeds. For example, the motor 112 may be configured to operate at a first speed, second speed, third speed, etc., each of which differ from one another. In some embodiments, the speed at which the motor is to rotate can be preset by an operator (e.g., a clinician or healthcare professional) and/or be determined or set based on the medical agent(s) to be mixed in the chamber of the device 100. In some embodiments, control of the motor 112 or actuating assembly 110 generally can be controlled via controls or a controller (not shown) on or separate from the device 100. For example, rotational speed, time of rotation, and/or other related parameters may be programmed into the controller, e.g., prior to the device 100 being loaded with a medical agent. In some embodiments, the actuating assembly 110 can also include a distal region 114 coupled to and extending distally from the actuating assembly 110. As shown in FIGS. 3A-3C, the distal region 114 can protrude in a distal direction from the rest of the actuating assembly 110, and can serve as a coupling element, e.g., to couple the actuating assembly 110 to the plunger assembly 120 or portions thereof.

The plunger assembly 120 can include a coupling member 130, a biasing member 140, a plunger 150, and a stirring member 160. With reference to FIGS. 3B and 3C, the coupling member 130 can include a first portion 132a (e.g., a proximal portion; FIG. 3B), a second portion 132b (e.g., a distal portion; FIG. 3B), a lip 134 (FIG. 3B) longitudinally between the first and second portions 132a, 132b, and an opening or slot 136 (FIG. 3C) extending longitudinally through the coupling member 130. The opening 136 may be configured (e.g., sized, shaped, or otherwise arranged) to receive other portions of the device 100. For example, the opening 136 may be configured to receive the distal region 114 of the actuating assembly 110 and a proximal end portion 161b of the stirring member 160. In doing so, the coupling member 130 can couple the actuating assembly 110 to the plunger assembly 120 or portions thereof. For example, the actuating assembly 110 and the stirring member 160 may be coupled to the coupling member 130 such that rotation or movement of the motor 112 of the actuating assembly 110 causes corresponding movement of the coupling member 130 and therein movement of the stirring member 160. In some embodiments, the coupling member 130 may secure the distal region 114 and/or the stirring member 160 disposed within the opening 136 via friction, an adhesive, or other connection means. The lip 134 can protrude axially or in a direction normal or angled to the longitudinal axis. The lip 134 may act as a backstop or support for the biasing member 140, thereby enabling the biasing member 140 to urge other portions of the plunger assembly 120 distally.

The biasing member 140 can include a spring, coiled or helical structure, or other resilient member configured to store and provide mechanical energy to an adjacent component. The biasing member 140 can be disposed distal to the coupling member 130 and proximal to plunger 150. In such embodiments, a proximal end of the biasing member 140 may be positioned directly against the lip 134 of the coupling member and around the second portion 132b, and a distal end of the biasing member 140 may be positioned against the plunger 150. The biasing member 140 can include any number of coils or turns so as to provide the necessary stiffness to the plunger 150, e.g., depending on the medical agent to be mixed by the device 100. As explained in additional detail elsewhere herein, the biasing member 140 is moveable between a compressed state to an uncompressed or less compressed state.

The plunger 150 (e.g., a plunger cap) is positioned distal to the biasing member 140 and is configured to be disposed within the housing 102. The plunger 150 can include a first portion 152 (e.g., a proximal portion; FIGS. 3B and 3C) having a first cross-sectional dimension, a second portion 154 (e.g., a distal portion; FIGS. 3B and 3C) distal to the first portion 152 and having a second cross-sectional dimension larger than the first cross-sectional dimension, and an opening or slot 158 (FIG. 3C) extending through the plunger 550. The opening 158 may be configured to slidably receive the stirring member 160 such that the stirring member 160 can move (e.g., rotatably move and/or longitudinally move) relative to the plunger 150 and opening 558. Additionally or alternatively, movement (e.g., longitudinal movement) of the stirring member 560 can occur and/or be controlled independently of longitudinal movement of the plunger 550. In some embodiments, a distal end portion of the opening 158 may be shaped to complement or correspond to a shape of the distal end portion 161a of the stirring member 160. In such embodiments, this may help seal the opening 158 to prevent fluid from leaking therethrough from a chamber 104 of the housing 102. The first cross-sectional dimension of the first portion 152 can be less than that of the biasing member 140 such that the distal end portion of the biasing member 140 can surround the first portion 152. The second portion 154 can include one or more concentric rings 156a, 156b, 156c (collectively referred to as “rings 156”) disposed around an outermost surface of the second portion 154. The rings 156 can comprise rubber, silicon, plastic, or other flexible material able to form a seal against the inner wall of the chamber 104 of the housing 102. When the plunger 150 is disposed within the housing 102, the plunger 150 can form a seal (e.g., an airtight seal) so as to prevent any fluid in the chamber 104 from leaking proximally therefrom. In some embodiments, a distalmost surface of the plunger 150 can have a conical or other shape that compliments or is similar to an internal surface of a distal end portion of a chamber 104 of the housing 102.

The stirring member 160 can be an elongate structure extending along the longitudinal axis of the device 100, and can include a distal end portion 161a and a proximal end portion 161b (FIGS. 3A and 3B). As described elsewhere herein, the proximal end portion 161b of the stirring member 160 can extend through the opening 158 of the plunger 150 and the biasing member 140 to be positioned within the coupling member 130. The distal end portion 161a can be positioned within the chamber 104 of the housing 102. In some embodiments, the distal end portion 161a (or distal terminus) of the stirring member 160 can have a conical or other shape that compliments or corresponds to an internal surface at the distal end of the chamber 104.

As previously described, the device 100 further includes the housing 102, which can comprise a chamber 104 (FIG. 3C) configured to hold or contain a fluid (e.g., the medical agent), and an exit port 106 (FIG. 3C) for transferring the fluid to another device (e.g., the connection assembly 30, the syringe 20, a catheter, etc.), e.g., for subsequent delivery to a patient. The housing 102 can comprise silicon, polymer, plastic, and/or other known materials used for syringes. As shown in FIG. 3C, a surface 108 at the distal end portion of the chamber 104 can include an angle or conical shape that generally corresponds to the surface at the distal end portion of the plunger 150. The corresponding surfaces of the plunger 150 and chamber 104 can enable the surfaces to mate, and thereby push substantially all of the fluid (e.g., mixed medical agent) in the chamber 104 out of the device 100.

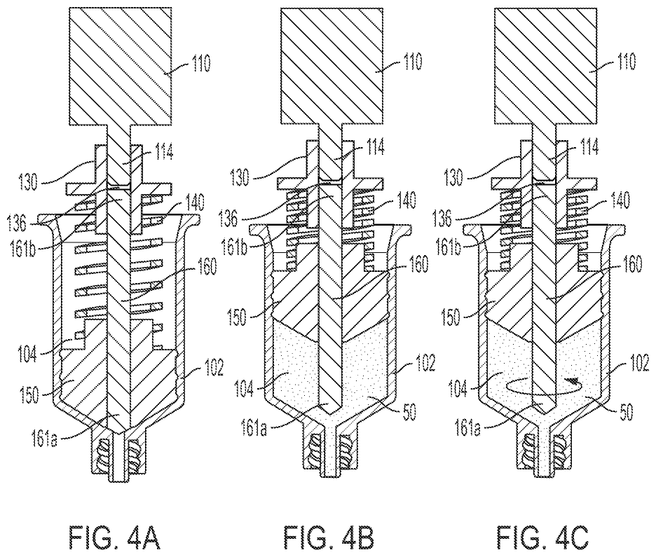

FIGS. 4A, 4B, and 4C show cross-sectional views of the mixing device 100 in different configurations, in accordance with embodiments of the present technology. The device 100 shown in FIG. 4A can correspond to a first position in which the plunger 150 is disposed at or toward the distal end portion of the chamber 104 such that no fluid is contained in the chamber 104. In the first configuration, the biasing member 140 can be in an uncompressed or unloaded state, or a less compressed or less loaded state relative to that of the biasing member 140 shown in FIG. 4B.

The device 100 shown in FIG. 4B can correspond to a second configuration in which the plunger 150 is disposed within the chamber 104 and spaced apart from the distal end portion of the chamber 104. Relative to the plunger 150 shown in FIG. 4A, the plunger 150 shown in FIG. 4B is moved proximally such that a fluid or medical agent 50 can be contained in the chamber 104. In some embodiments, the fluid 50 can be drawn (e.g., from a container) into the chamber 104 by urging the plunger 150 or plunger assembly 120 (FIG. 3A) proximally. In the second configuration, the biasing member 140 can be in a compressed or loaded state, or a more compressed or more loaded state, relative to that of the biasing member 140 shown in FIG. 4A.

The device 100 shown in FIG. 4C has the actuating assembly 110 (or motor 112 (FIGS. 3A-3C)) in an active state or turned “ON”, thereby causing movement of the stirring member 160, e.g., via movement of the coupling member 130. As described elsewhere herein, the stirring member 160 can rotate at a plurality of different speeds and/or for different periods of time, each of which may be predetermined, e.g., based on properties (e.g., composition) of the fluid 50 to be mixed. For example, the stirring member 160 may rotate at a first speed for a first period of time, and subsequently at a second, different (e.g., faster or slower) speed for a second period of time. This may be done to ensure the fluid 50 is adequately mixed and/or to ensure the fluid 50 remains adequately mixed, e.g., until the mixed fluid 50 is transferred to a syringe, catheter, or other device for delivery to a patient.

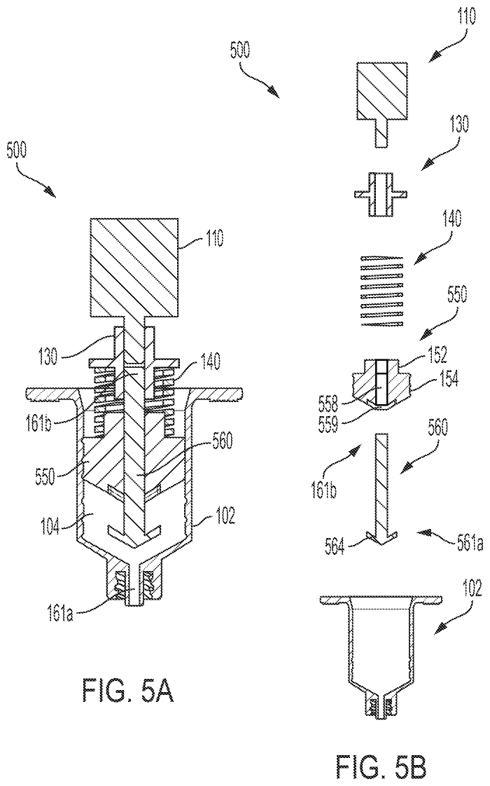

FIGS. 5A and 5B show cross-sectional views of a device 500 for mixing a medical agent, in accordance with embodiments of the present technology. The mixing device 500 can include features and functionality similar or identical to those features and functionality of the device 100 previously described, unless indicated otherwise below. As shown in FIGS. 5A and 5B, the actuating assembly 110, the coupling member 130, the biasing member 140, and housing 102 are generally the same as previously described. The device 500 can further include a plunger 550 and a stirring member 560, which generally includes many of the features and functionality of the plunger 150 and stirring member 160 respectively previously described.

The plunger 550 (e.g., a plunger cap) is positioned distal to the biasing member 140 and is configured to be disposed within the housing 102. With reference to FIG. 5B, the plunger 550 can include a first portion 152 (e.g., a proximal portion) having a first cross-sectional dimension, a second portion 154 (e.g., a distal portion) distal to the first portion 152 and having a second cross-sectional dimension larger than the first cross-sectional dimension, and an opening or slot 558 extending through the plunger 550. The opening 558 may be configured to slidably receive the stirring member 560 such that the stirring member 560 can longitudinally and/or rotationally move relative to the plunger 550 and/or opening 558. Additionally or alternatively, longitudinal movement of the stirring member 560 can occur and/or be controlled independently of longitudinal movement of the plunger 550. In some embodiments, a distal end portion 559 of the opening 558 may be shaped to complement or correspond to a shape of the distal end portion 561a of the stirring member 560. In such embodiments, this may help seal the opening 558 to prevent fluid from leaking therethrough from the chamber 104 (FIG. 5A). The first cross-sectional dimension of the first portion 152 can be less than that of the biasing member 140 such that the distal end portion of the biasing member 140 can surround the first portion 152. When the plunger 550 is disposed within the housing 102, the plunger 550 can form a seal (e.g., an airtight seal) so as to prevent any fluid in the housing 102 from leaking proximally therefrom. In some embodiments, a surface at the distal end (or distal terminus) of the plunger 550 can have a conical or other shape that compliments or is similar to an internal surface of a distal end portion of the chamber 104 of the housing 102.

The stirring member 560 can be an elongate structure extending along the longitudinal axis of the device 500, and can include a distal end portion 561a and a proximal end portion 161b. As described elsewhere herein, the proximal end portion 161b of the stirring member 160 can extend through the opening 558 of the plunger 550 and the biasing member 140 to be positioned within the coupling member 130. The distal end portion 561a can be positioned within the chamber 104 of the housing 102. The stirring member 560 can include a flared portion 564 (e.g., wings) at the distal end portion 561a that protrude axially from the elongate structure. In some embodiments, the stirring member 560 may include other flared portions, e.g., at an intermediate portion of the elongate structure between the distal and proximal ends 561a, 561b. Additionally or alternatively, the flared portion 564 and/or the distal end portion 561a (or distal terminus) of the stirring member 560 can have a conical or other shape that compliments or is similar to an internal surface of the chamber 104.

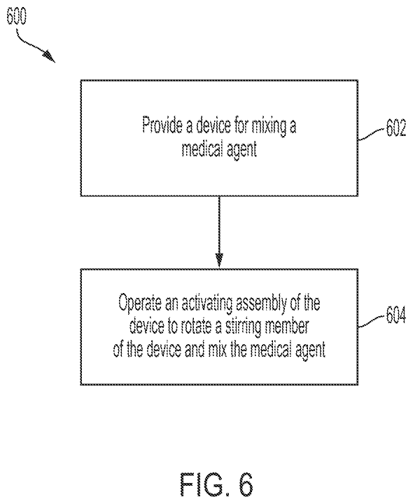

FIG. 6 is a flow diagram of a method 600 for mixing a medical agent, in accordance with embodiments of the present technology. The method 600 can comprise providing a device for mixing a medical agent (process portion 602). The device can be the mixing device 100 or 500 described herein with reference to FIGS. 1A-5S. As such, the device can include an actuating assembly (e.g., the actuating assembly 110), a plunger assembly (e.g., the plunger assembly 120), and a housing (e.g., the housing 102) or portions thereof. The method 600 can further comprise operating the actuating assembly of the device to rotate the stirring member of the device (process portion 604), and thereby mix any medical agent contained in the housing. Operating the actuating assembly can comprise initiating the actuating assembly to an active or “ON” state in which the motor of the actuating assembly begins to rotate at a predetermined speed. In some embodiments, prior to operating the actuating assembly, the method 600 may comprise drawing the medical agent (e.g., from a container) into a chamber (e.g., the chamber 104) of the housing of the device, e.g., by withdrawing the plunger assembly proximally relative to the housing.

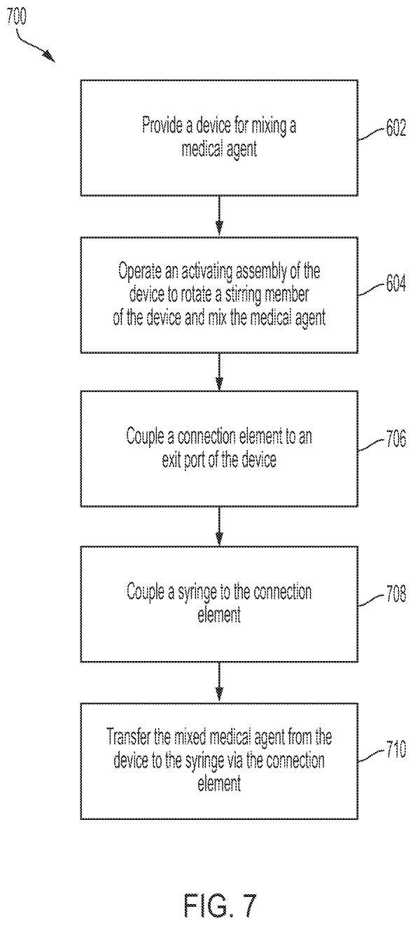

FIG. 7 is a flow diagram of a method 700 for mixing a medical agent, in accordance with embodiments of the present technology. The method 700 can comprise process portions 602 and 604, and can further comprise coupling a connection assembly (e.g., the connection assembly 30) to an exit port of the mixing device (process portion 706). Coupling the connection assembly to the exit port of the mixing device can occur before or after process portion 604. In some embodiments, the method 700 can optionally include subsequently adjusting a flow control element (e.g., the flow control element 32) of the connection assembly to create a flow path from the device. For example, adjusting the flow control element can create a first flow path between the device and the atmosphere that allows trapped air in the chamber of the device to escape.

The method 700 can further comprise coupling a syringe (e.g., the syringe 20) to the connection assembly 30 (process portion 708), and transferring the medical agent from the device to the syringe via the connection assembly (process portion 710). Coupling the syringe to the connection assembly may occur before or after process portion 706. In some embodiments, transferring the medical agent from the device to the syringe can comprise adjusting the flow control element to create a second flow path from the device to the syringe, and then urging the plunger of the device distally such that the medical agent is pushed from the device toward the connection assembly and syringe. After being transferred to the device, the flow control element may be adjusted to close the second flow path, and the syringe may be decoupled from the connection assembly.

### III. Conclusion

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Although many of the embodiments are described above with respect to systems, devices, and methods for manufacturing core members for use with medical devices, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-7.

I/we claim:

1. A device for mixing a medical agent, the device comprising:
a housing defining a chamber configured to contain a fluid;
a plunger assembly configured to be received by the chamber, the plunger assembly comprising a plunger and a stirring member extending along a longitudinal axis, the stirring member being rotatably coupled to the plunger and having a distal portion disposed within the chamber of the housing; and
an actuating assembly operably coupled to the stirring member, the actuating assembly having an active state in which the actuating assembly causes the distal end portion of the stirring member to rotate at a predetermined speed.

2. The device of claim 1, wherein the actuating assembly comprises a motor disposed along the longitudinal axis, and wherein rotation of the motor causes the distal end portion of the stirring member to rotate.

3. The device of claim 2, wherein the motor is a step motor configured to rotate at a plurality of speeds including a first speed, and a second speed different than the first speed.

4. The device of claim 1, further comprising a coupling member coupled directly to the actuating assembly and the stirring member such that rotation of the actuating assembly causes corresponding rotation of at least one of the coupling member and the stirring member.

5. The device of claim 1, further comprising a biasing member disposed between the plunger and the actuating assembly, the biasing member urging the plunger distally toward the housing.

6. The device of claim 5, wherein the plunger is movable relative to the housing from a first position adjacent a distal portion of the chamber to a second position spaced apart from the distal portion of the chamber, wherein, when the biasing member is in a first state when the plunger is in the first position and a second state when the plunger is in the second position, and wherein the biasing member in the second state is more compressed than the biasing member in the first state.

7. The device of claim 5, further comprising a coupling member having a proximal end and a distal end opposite the proximal end, the coupling member receiving a portion of the actuating assembly at the proximal end and a portion of the stirring member at the distal end, wherein the biasing member is disposed between the coupling member and the plunger such that the biasing members acts against the coupling member and the plunger to urge the plunger distally.

8. The device of claim 1, wherein the stirring member is longitudinally moveable relative to the plunger.

9. The device of claim 1, wherein the plunger a slot extending along the longitudinal axis, and wherein the stirring member extends through the slot and is longitudinally moveable relative to the plunger.

10. The device of claim 1, further comprising an exit port at a distal end of the housing, the exit port being configured to be coupled to a connection assembly via luer lock connection fittings.

11. The device of claim 1, wherein the stirring member comprises a flared portion at the distal portion of the stirring member, the flare portion extending at an angle relative to the longitudinal axis.

12. A medical device system, comprising:
a mixing device including—
a housing defining a chamber configured to contain a fluid and having a longitudinal axis;
a plunger assembly configured to be received by the chamber, the plunger assembly comprising a plunger and a stirring member extending along the longitudinal axis, the plunger being rotatably coupled to the plunger, the stirring member having a distal portion disposed within the chamber of the housing; and
an actuating assembly operably coupled to the stirring member, the actuating assembly having an active state in which the actuating assembly causes the distal end portion of the stirring member to rotate at a predetermined speed.

13. The system of claim 12, further comprising a connection assembly including a first port coupled to an exit port of the housing, and a second port configured to be exposed to atmosphere, wherein a flow path between the first and second ports is configured to release trapped air within the chamber.

14. The system of claim 12, further comprising a syringe and a connection assembly, the connection assembly including a first port coupled to an exit port of the housing, and a second port coupled to the syringe, wherein the chamber of the device and the syringe are in fluid communication with one another via a flow path extending between the first and second ports.

15. The system of claim 12, further comprising a connection assembly including:
a flow control element,
a first port coupled to an exit port of the housing,
a second port configured to be exposed to the atmosphere, and
a third port configured to be coupled to a syringe,
wherein movement of the flow control element creates a flow path between the first port and one of the second port or the third port.

16. A method for mixing a medical agent, the method comprising:
providing a mixing device including—
a housing defining a chamber containing a fluid and having a longitudinal axis;
a plunger assembly at least partially disposed within the chamber, the plunger assembly including a plunger and a stirring member rotatably coupled to the plunger, the stirring member disposed within the chamber of the housing such that a distal portion of the stirring member is proximate the fluid; and
an actuating assembly operably coupled to the stirring member, the actuating assembly having an active state in which the actuating assembly causes the distal end portion of the stirring member to rotate at a predetermined speed; and
operating the actuating assembly such that the stirring member mixes the fluid.

17. The method of claim 16, wherein the mixing device is in a first configuration in which the plunger is positioned at a distal portion of the chamber, the method further comprising urging the plunger proximally along the longitudinal axis toward the actuating assembly, thereby compressing a biasing member of the device.

18. The method of claim 16, wherein the actuating assembly comprises a motor, and wherein operating the actuating assembly comprises activating the motor to rotate at a predetermined speed for a predetermined period of time.

19. The method of claim 16, further comprising:
coupling a first port of a connection assembly to an exit port of the housing; and
enabling trapped air in the chamber to be released via a second port of the connection assembly.

20. The method of claim 16, further comprising:
coupling a first port of a connection assembly to an exit port of the housing;
coupling a syringe to a second port of the connection assembly; and
transferring the fluid from the chamber to the syringe via a path include the first and second ports of the connection assembly.
